(19) United States
(12) Patent Application Publication
(10) Pub. No.: US 2021/0325032 A1
(43) Pub. Date: Oct. 21, 2021

(54) Flesh-Illuminating Device

(71) Applicants: Aleksandar NANEV, Saint Petersburg, FL (US); Project Leah, LLC, Saint Petersburg, FL (US)

(72) Inventor: Aleksandar Nanev Nanev, Saint Petersburg, FL (US)

(73) Assignee: Project Leah, LLC, Saint Petersburg, FL (US)

(21) Appl. No.: 16/338,479

(22) PCT Filed: Nov. 19, 2018
(86) PCT No.: PCT/US18/61891

§ 371 Date: Mar. 31, 2019

Related U.S. Application Data
(60) Provisional application No. 62/588,241, filed on Nov. 17, 2017.

(51) Int. Cl. F21V 33/00; G06F 3/16; F21V 19/00; F21V 23/04; F21S 9/02; F21V 11/00; F21S 10/06

(52) U.S. Cl. CPC F21V 33/0008; G06F 3/16; F21V 19/001; F21S 10/06; F21S 9/02; F21V 11/00; F21V 23/04

(57) ABSTRACT

A flesh-illuminating device includes: a light source configured to generate light usable to illuminate flesh of a user; a shape holder operably connected to the light source, the shape holder configured to hold the light source in place so that the light source can illuminate the user's flesh; a user interface operably connected to the light source, the user interface configured to allow the user to control the light source; and a power source operably connected to the light source, the power source configured to provide power to the flesh-illuminating device.

### PRIORITY CLAIM

The present application claims the priority benefit of U.S. provisional patent application No. 62/588,241 filed Nov. 17, 2017 and entitled “Device for Lighting Flesh,” the disclosure of which is incorporated herein by reference.

### SUMMARY

A flesh-illuminating device includes: a light source configured to generate light usable to illuminate flesh of a human user; a shape holder operably connected to the light source, the shape holder configured to hold the light source in place so that the light source can illuminate the user's flesh; a user interface operably connected to the light source, the user interface configured to allow the user to control the light source; and a power source operably connected to the light source, the power source configured to provide power to the flesh-illuminating device.

A flesh-illuminating device includes: a light source configured to generate light usable to illuminate flesh of a human user; a shape holder operably connected to the light source, the shape holder configured to hold the light source in place so that the light source can illuminate the user's flesh; a user interface operably connected to the light source, the user interface configured to allow the user to control the light source; a power source operably connected to the light source, the power source configured to provide power to the flesh-illuminating device; and a sensor configured to determine when the flesh-illuminating device does one or more of contact skin of the user and come within a predetermined distance of the user, wherein the flesh-illuminating device provides near 360 degrees of visibility of the illuminated flesh, wherein the flesh-illuminating device is configured to enter an activated mode within a predetermined distance from a location of interest, wherein the location of interest comprises one or more of a crosswalk, an intersection, a highway, and a major road, wherein the activated mode comprises one or more of a stronger light, a weaker light, a blinking light and another attention-drawing mode of the light source, wherein the activated mode is configured to attract attention of a driver of a motor vehicle, wherein the light source is configured to activate based on an activation criterion received from a user, the activation criterion comprising one or more of a location of the flesh-illuminating device, a distance of the flesh-illuminating device from the user, a detection of ambient light, a date, a location, and a time of day, wherein the flesh-illuminating device is configured to send a user a reminder to turn the light source on for one or more of safety reasons, user pre-set preferences, and other reasons, wherein the flesh-illuminating device is further configured to receive a user preference regarding a user activity that disables the flesh-illuminating device, the user activity comprising one or more of running, bicycling, hiking, skating, skiing, participating in a designated sport, and walking.

### DESCRIPTION OF THE DRAWINGS

The accompanying drawings provide visual representations which will be used to more fully describe various representative embodiments and can be used by those skilled in the art to better understand the representative embodiments disclosed herein and their inherent advantages. In these drawings, like reference numerals identify corresponding elements.

FIG. 1 is a drawing of a flesh-illuminating device.

FIGS. 2A-2B are a set of two drawings of a flesh-illuminating device comprising a sound-emitting device configured to emit sound

FIGS. 3A-3B are a set of two drawings of a flesh-illuminating device designed to be worn around an ear of a user.

FIGS. 4A-4C are a set of three drawings of a flesh-illuminating device designed to be worn as a band around a neck of a user.

FIG. 5 are a set of two drawings of a flesh-illuminating device designed to be worn as a band around a user's neck.

FIG. 6 is a drawing of a flesh-illuminating device designed to be worn inside the nostril of a user.

FIG. 7 is a drawing of a flesh-illuminating device designed to be worn as an ear gauge on an earlobe of a user.

FIGS. 8A-8B are a set of two drawings of a flesh-illuminating device designed to be worn as a necklace on a neck of a user.

### DETAILED DESCRIPTION

Embodiments of the invention relate in general to a device and method for lighting flesh. Further embodiments of the invention relate to a device and method for lighting flesh, using a human body as a light transmitting medium. Other embodiments of the invention relate to a device and method for lighting flesh, using a human body as a light diffusing medium. Still further embodiments of the invention relate to a device and method for lighting flesh, using a body as a light transmitting environment. Yet additional embodiments of the invention relate to a device and method for lighting flesh, using a body as a light diffusing environment.

A flesh-illuminating device comprises a light source configured to generate light usable to illuminate flesh of a human user. The flesh-illuminating device further comprises a shape holder operably connected to the light source, the shape holder configured to hold the light source in place so that the light source can illuminate the user's flesh. The flesh-illuminating device further includes a user interface operably connected to the light source, the user interface configured to allow the user to control the light source. For example, the user interface is directly connected to the light source. For example, the user interface is directly connected to the light source through a wire. For example, the user interface is connected to a controller, and the controller is directly connected to the light source. The flesh-illuminating device further includes a power source operably connected to the light source, the power source configured to provide power to the flesh-illuminating device.

Additional embodiments of the invention comprise a light source configured to generate light usable to illuminate flesh of a human user; and a shape holder operably connected to the light source, the shape holder configured to hold the light source in place so that the light source can illuminate the user's flesh. For example, one or more of the light source, the control and the power source can be omitted from embodiments of the invention. For example, a user provides one or more of the light source, the control and the power source.

Additional embodiments of the invention relate to a device and method for lighting flesh, using a human user's ear as a light transmitting medium. Other embodiments of the invention relate to a device and method for lighting flesh, using a human ear as a light diffusing medium.

According to additional embodiments of the invention, the flesh-illuminating device can activate based on one or more activation criterion. For example, the activation criterion comprises one or more of a location of the flesh-illuminating device, a distance of the flesh-illuminating device from the user, a detection of ambient light, a date, a location, and a time of day. For example, the flesh-illuminating device turns itself on as dusk approaches and turns itself off as dawn approaches. For example, the light sensor comprises an ambient light sensor. For example, the flesh-illuminating device comprises earphones configured to send the user a reminder to turn the light source on for one or more of safety reasons, pre-set user preferences, and other reasons. For example, the flesh-illuminating device is configured to receive a user preference regarding a user activity that enables the flesh-illuminating device. For example, the enabling user activity comprises one or more of running, bicycling, skating, skiing, participating in a designated sport, and walking.

For example, the controller receives the activation criterion from the user. For example, a smart device to which the controller is connected receives the activation criterion from the user.

For example, the activation criterion is determined at the flesh-illuminating device by the controller. For example, the activation criterion is determined by a smart device to which the controller is connected. For example, the smart device comprises one or more of a smartphone, a music player, a video player, and another smart device.

For example, the user comprises one or more of a pedestrian, a bicyclist, a motorcycle operator, and a child. For example, when activated, the flesh-illuminating device provides nearly 360 degrees of visibility of the illuminated flesh.

For example, the flesh-illuminating device is configured to enter an activated mode within a predetermined distance from a location of interest. For example, the location of interest comprises one or more of a crosswalk, an intersection, a highway, a major road, and another location of interest. For example, the flesh-illuminating device determines a position of the user. For example, the flesh-illuminating device determines a position of the user using navigation data. For example, the navigation data comprises data from one or more of the Global Positioning System (GPS) and the Global Navigation Satellite System (GLONASS). For example, the flesh-illuminating device determines a position of the user in conjunction with a smart device. For example, as the user approaches the location of interest, the flesh-illuminating device activates. For example, activation comprises one or more of turning on the light source, turning off the light source, and changing a mode of the light source to an activated mode.

For example, the activated mode comprises one or more of a stronger light, a weaker light, a blinking light, and another attention-drawing mode. For example, the activated mode is configured to attract attention of a driver of a motor vehicle. For example, the activated mode can be suppressed when the user is in a motor vehicle.

Further embodiments of the invention relate to a device and method for lighting flesh, using as a light transmitting medium one or more of a human ear, a human ear root, a human ear lobe, an earring worn by a human, a human ear edge, a human nose, a human mouth, a human hand, a human finger, a human fingernail, a human toenail, a human foot, a human toe, a human head, a human neck, a human breast, human buttocks, a human buttocks cheek, a human cheek, a human stomach, a human belly button, a human leg, a human ankle, a bra worn by a human, a belt worn by a human, a wristband worn by a human, a wristwatch worn by a human, a sandal worn by human, a shoe worn by human, another clothing item worn by a human, a human body, human flesh, a human lip, and another human body part.

Still further embodiments of the invention relate to a device and method for lighting flesh, using as a light diffusing medium one or more of a human ear, a human ear root, a human ear lobe, an earring worn by a human, a human ear edge, a human nose, a human mouth, a human hand, a human finger, a human fingernail, a human toenail, a human foot, a human toe, a human head, a human neck, a human breast, human buttocks, a human buttocks cheek, a human cheek, a human stomach, a human belly button, a human leg, a human ankle, a bra worn by a human, a belt worn by a human, a wristband worn by a human, a wristwatch worn by a human, a sandal worn by human, a shoe worn by human, another clothing item worn by a human, a human body, human flesh, a human lip, and another human body part.

Additional embodiments of the invention relate to a device and method for lighting flesh, using as one or more of a light transmitting medium and a light diffusing medium one or more of an animal ear, an animal ear root, an animal ear lobe, an earring worn by an animal, an animal ear edge, an animal nose, an animal mouth, an animal hand, an animal finger, an animal fingernail, an animal toenail, an animal foot, an animal toe, an animal neck, an animal head, an animal breast, animal buttocks, an animal buttocks cheek, an animal cheek, an animal stomach, an animal belly button, an animal ankle, a wristband worn by an animal, an animal paw, an animal hoof, an animal ankle, an animal tail, an animal horn, an animal tusk, an animal antler, another clothing item worn by an animal, an animal body, animal flesh, an animal lip, and another animal body part.

According to further embodiments of the invention, the flesh-illuminating device comprises a light source. For example, the light source comprises one or more of a light-emitting diode (LED), an organic light-emitting diode (OLED), a micro-LED, a nano-LED, an LED filament, a laser, a fiber optic light source, a glowing fiber optic light source, a filament light bulb, a non-filament light bulb, a fluorescent light source, a future light source, bioluminescence, chemiluminescence, phosphorescence, radioluminescence, photoluminescence, electroluminescence, and another light source.

According to still further embodiments of the invention, the flesh-illuminating device comprises a plurality of light sources. For example, the flesh-illuminating device comprises a plurality of addressable light sources. For example, the flesh-illuminating device comprises a plurality of addressable, individually controllable, small, light-emitting mini-sources, for example, one or more of an LED and a micro-LED.

For example, the flesh-illuminating device uses a light source external to a human body. For example, the flesh-illuminating device uses a light source internal to a human body. For example, the flesh-illuminating device uses a light source external to a human body and also uses a light source internal to a human body. For example, the flesh-illuminating device uses a light source external to a human body that extends into the human body.

For example, one or more of the addressable light sources can be individually controlled. For example, one or more of the addressable light sources can be individually controlled by one or more of a user, a device, a program, and an artificial intelligence (AI). For example, one or more of the addressable light sources can be turned on by the one or more of a user, a device, a program, and an AI. For example, one or more of the addressable light sources can be turned off by the one or more of a user, a device, a program, and an AI. For example, one or more of the addressable light sources can be dimmed by the one or more of a user, a device, a program, and an AI. For example, one or more of the addressable light sources can be brightened by the one or more of a user, a device, a program, and an AI. For example, one or more of the addressable light sources can be locally dimmed on by the user. For example, one or more of the addressable light sources can be locally dimmed off by the user.

For example, the light can be locally dimmed in parts or sections where the flesh-illuminating device is in contact with the skin. For example, local dimming renders the lighting more even by reducing lighting of areas where the flesh-illuminating device touches the skin. For example, the light can be locally dimmed in parts or sections where the ear is too thin, so the light is more even. Local dimming allows the ear to be illuminated more evenly. The local dimming comprises one or more of local dimming on and local dimming off. Alternatively, or additionally, local dimming compensates for user ears having one or more of a different shape and a different length. Alternatively, or additionally, local light can be one or more of switched on, switched off, and dimmed at an end of the ear close to the earlobe to prevent one or more of illumination outside of the ear and illumination over the ear.

For example, using the addressable light source, the user can change a color of illuminating light.

For example, the flesh-illuminating device can do one of more of diffuse, emit, and glow light.

For example: using an AI, which performs needed calculations and provides the results to the user, the user can look through a smart device's camera and perform the local dimming. For example, the user can perform the calculation manually by one or more of looking in a mirror and looking at a device's screen. For example, the user can perform a manual local adjustment using an interface of a smart device. For example, the user can perform the calculation using an algorithm.

For example, the local dimming can facilitate a visual effect, for example, one or more of a running light, a wave, and a heartbeat rhythm.

For example, the flesh-illuminating device uses light in one or more of a visible wavelength, an ultraviolet wavelength, and an infrared wavelength. For example, the flesh-illuminating device uses visible light of any color in the visible spectrum, for example, one or more of white light, red light, blue light, green light, and another color of visible light. For example, during cold weather, the infrared can additionally be used to warm a user's ears.

For example, the flesh-illuminating device uses an LED configured to radiate light after it passes through a medium and excites the medium into emitting visible light. For example, the medium comprises one or more of phosphor, remote phosphor at a distance from the LED and another light-emitting medium. For example, the LED emits light having a wavelength between approximately 100 nanometers (nm), and approximately 400 nm, that is, light in a range of wavelengths associated with the ultraviolet spectrum. For example, this light can be used to excite one or more of the phosphor and the remote phosphor using the effect of photoluminescence. For example, the phosphor combines to emit light of a given overall wavelength, for example, red light having a peak wavelength of approximately 650 nm.

For example, the phosphor excites light at different wavelengths so that the overall color of the light is white. A majority of the white light is one or more of absorbed as heat and reflected.

The flesh-illuminating device further comprises a power source. The flesh-illuminating device further comprises a user interface configured to allow the user to control the light source. Optionally, the flesh-illuminating device further comprises a power recipient. For example, the power recipient comprises one or more of an inductive coil and another power recipient. For example, one or more of the power source, the user interface, and the power recipient is integrated into the human body.

For example, the flesh-illuminating device is operably connected to one or more of a power source, a light source, and a control device. For example, the flesh-illuminating device is directly connected to the one or more of a power source, a light source, and a control device.

For example, the flesh-illuminating device further comprises one or more of earbuds, headphones, and another sound-emitting device configured to generate sound. For example, the sound-emitting device is itself configured to be illuminated. For example, the sound-emitting device is itself configured to light flesh. Embodiments of the invention comprise a combination of the flesh-illuminating device and the sound-emitting flesh-illuminating device.

For example, the sound-emitting device is operably connected to one or more of a power source, a light source, and a control device. For example, the sound-emitting device is directly connected to the one or more of a power source, a light source, and a control device.

For example, the sound-emitting device is detachable from the rest of the flesh-illuminating device. For example, the detachable sound-emitting device enables use of the flesh-illuminating device just for illumination purposes so that there is no interference with the user's hearing. For example, the flesh-illuminating device is mounted on the sound-emitting device.

For example, the flesh-illuminating device further comprises a speaker configured to create sound effects that operate in conjunction with the light effects. Alternatively, or additionally, another device comprises the speaker. For example, a smartphone comprises the speaker. For example, the speaker generates sound effects that are one or more of spooky, scary, and funny.

For example, the flesh-illuminating device is configured to function as a visual alarm signaling when a time of interest has arrived, for example, a time the user wishes to get out of bed, a time the user needs to work, a time the user needs to contact a colleague, and another time of interest. For example, the flesh-illuminating device is configured to function as a visual alarm configured to receive an alert from device. For example, the alert is silent. For example, the alert is audible. For example, the flesh-illuminating device comprises a mobile phone. For example, the flesh-illuminating device comprises a tablet.

For example, the flesh-illuminating device is configured to alert the user when a designated condition occurs. For example, the designated condition comprises one or more of an incoming telephone call, a triggering of an alarm in a vehicle, a triggering of a house alarm, and another designated condition.

For example, the flesh-illuminating device is operably connected to an adapter, and the adapter is in turn operably connected to the one or more of a power source, a light source, and a control device. For example, the power source uses one or more of optical charging, protruded electrodes, wireless charging, and another form of charging.

For example, the flesh-illuminating device comprises a sound-emitting device, with at least one of the flesh-illuminating device and the sound-emitting device being operably connected with a headphone jack. For example, the sound-emitting device is directly connected with the headphone jack. For example, the sound-emitting device is directly connected with the headphone jack via a cable. For example, one or more of a power source, a light source, and a control device is operably connected to both the sound-emitting device and the headphone jack. For example, the one or more of a power source, a light source, and a control device is directly connected to the sound-emitting device, the one or more of a power source, a light source, and a control device also being directly connected to the headphone jack. For example, the headphone jack is used to transfer part of the power used by the light source. For example, one or more of a separate intermediate battery and the user interface provides the power used by the light source.

For example, the flesh-illuminating device is operably connected to the controller via one or more of a uniform serial bus (USB), another wired interface, Bluetooth, Zigbee, WiFi, another wireless interface, and a direct connection. For example, the interface comprises a Lightning interface manufactured by Apple Computer (www.apple.com) of Cupertino, Calif. For example, a controller comprises one or more of a computer, a laptop, a desktop computer, a tablet, a mobile phone, an MP3 player, another music player, a smart device, and another controller.

Alternatively, or additionally, a master controller controls multiple flesh-illuminating devices. For example, a plurality of devices for lighting flesh is provided, the flesh-illuminating device being configured for use at one or more of a concert, a club, an event, a party, and a similar event. For example, at least two of the plurality of devices may be controlled by the master controller. For example, at least two of the plurality of devices may be controlled by the master controller, so as to provide substantially synchronized control of at least two of the flesh-illuminating devices for lighting flesh. For example, a device can be triggered by one or more of a selected genre of music, a selected rhythm, and another selection criterion.

For example, one or more of the flesh-illuminating device and the sound-emitting flesh-illuminating device can be used to take a photograph of oneself (a “selfie”). For example, the selfie is a selfie comprising an image only of the person taking the photograph. For example, the selfie is a selfie comprising an image of the person taking the photograph and one or more additional persons. For example, the selfie is taken using one or more of a camera, a mobile phone, a tablet, and another image-capturing device.

For example, the one or more of the flesh-illuminating device and the sound-emitting flesh-illuminating device can be operating in approximate synchronization with the image-capturing device camera. That is, the flesh-illuminating device can suddenly (or non-suddenly) be turned on and the image-capturing device used to capture an image of a previously unsuspecting person. For example, the flesh-illuminating device can suddenly (or non-suddenly) be turned on and the image-capturing device used to capture a surprise picture of a person with an illuminated ear. For example, the flesh-illuminating device can suddenly (or non-suddenly) be turned on while a song is approximately simultaneously played and the image-capturing device used to capture a surprise picture of a person with an illuminated ear who is listening to the song.

For example, the flesh-illuminating device comprises a heart pulse sensor configured to sense a heartbeat of a user. For example, the flesh-illuminating device lights up in approximate synchronization with the user's heartbeat. Alternatively, or additionally, the flesh-illuminating device is configured to receive a signal from a heart pulse sensor located external to the flesh-illuminating device so that the flesh-illuminating device lights up in approximate synchronization with the user's heartbeat. For example, one or more of a smart phone, a smart watch, and another device comprises the external heart pulse sensor. Alternatively, or additionally, the flesh-illuminating device is configured to receive a false heartbeat signal from a false heartbeat generator located external to the flesh-illuminating device. The false heartbeat has no connection to a heartbeat of any actual living being. Alternatively, or additionally, the flesh-illuminating device generates the false heartbeat even without a real pulse signal from any device, as a standalone feature.

Similarly to the pulse effect, any other visual effect of interest can be provided by the flesh-illuminating device, for example, flashing, blinking, running lights, a strobe effect, a dimming, a local dimming, pulsating by/with the rhythm of the song played and the like.

For example, the AI can use the flesh-illuminating device to create one or more of an alert and a signal. For example, the AI comprises one or more of Siri, Alexa, Cortana, and another AI.

For example, a device notification can cause the flesh-illuminating device to light flesh. For example, the flesh-illuminating device can be triggered by one or more of an incoming telephone call, a voicemail, an incoming text message, an incoming electronic mail (email) message, and another device notification. For example, one or more of an ear, an arm, a leg, a foot, and a wrist of a human is illuminated by the flesh-illuminating device when the user's phone rings.

For example, while wearing the flesh-illuminating device, the user can point a smart device at the user's face and thereby cause the AI to detect one or more of the user's face, the user's ear, and another feature of the user's face or body. The AI will then adjust the light according to the detected feature. For example, the AI adjusts the light so that a user's ears are evenly illuminated. For example, the program adjusts the light.

For example, the flesh-illuminating device comprises a microphone. For example, the microphone comprises one or more of a built-in microphone and a standalone microphone. For example, the flesh-illuminating device may be activated by one or more a scary laugh, a sound of crying, another sound, and another activation trigger. For example, the flesh-illuminating device is activated by a notification to one or more of a computer, a laptop, a desktop computer, a tablet, a mobile phone, an MP3 player, another music player, and another controller. For example, the flesh-illuminating device is activated by one or more of a ringing of a telephone, an incoming phone call, a voicemail message, a text message, and another notification to the one or more of a computer, a laptop, a desktop computer, a tablet, a mobile phone, an MP3 player, another music player, and another controller. For example, the activation of the flesh-illuminating device triggers illumination of one or more of a human body part and an animal body part. For example, the activation of the flesh-illuminating device triggers illumination of the user's ear and another body part of the user.

For example, the invention further comprises a cord that is configured to be illuminated.

For example, the flesh-illuminating device is activated by one or more preselected triggers. For example, the trigger comprises one or more of a user's anger, a scream, a growl, a laugh, a hum, a preselected emotion, a voice activation, and another trigger.

For example, the flesh-illuminating device illuminates the user's nose and further comprises a clip to hold the flesh-illuminating device to the user's nose. For example, the clip holds the flesh-illuminating device to the user's nostrils.

For example, the flesh-illuminating device is comprised in a nose pad of the user's glasses. For example, the flesh-illuminating device is used to illuminate the user's nose. For example, the light source directs light into the user's nose, illuminating it. For example, the light source is mounted on the glasses, on an extension cord for the glasses, and on temple tips of the glasses.

For example, the flesh-illuminating device is placed between the user's lips and teeth. For example, the light source direct light away from the user's body so as to illuminate one or more of the lips and the cheeks.

For example, the light source can be placed inside a user's headwear, illuminating the user's flesh where the hat contacts the flesh.

For example, the light source makes a full circle around the user's head.

For example, the flesh-illuminating device is placed on the edge of the user's bra, preferably but not necessarily on an edge of a cup of the bra, so that the user's breast is illuminated. For example, the flesh-illuminating device is positioned under the bra cup. For example, the flesh-illuminating device is positioned over the user's nipple, so that the breast is illuminated from the nipple out.

For example, a medallion comprises the flesh-illuminating device. For example, the medallion is configured to be placed between the user's breasts, illuminating them. For example, a patch comprises the flesh-illuminating device. For example, the patch adheres to the user's skin with one or more of a washable glue, another self-sticking, removable material, and a suction cup. For example, a sticker comprises the flesh-illuminating device. For example, the flesh-illuminating device comprises netting, so that the user's skin is illuminated under the netting. For example, the netting also has a toy spider on it.

For example, the flesh-illuminating device is mounted on a side of small headphones that do not fully cover a user's ear so as to illuminate uncovered sections of the use's ear.

For example, the sound-emitting device can be used at night or at other times of reduced visibility by one or more of a runner, a jogger, a bicyclist, and another exercising user. The sound-emitting device satisfies functions of one or more of headphones, signaling, and fashion.

For example, the flesh-illuminating device further comprises a shadowing device configured to reduce one or more of direct reflection of light from the user's flesh and direct light from the light source. For example, the shadowing device is physically connected to one or more of the light source and the shape holder. For example, the shadowing device is configured to substantially prevent reflection of light from the user's flesh. For example, the shadowing device is configured to prevent reflection of light from the user's ear. The shadowing device may thereby prevent light from dazzling a person behind the user.

For example, the shadowing device comprises a fin. For example the fin comprises one or more of a membrane, foam, silicone, viscoelastic silicone rubber, sponge, leather, and another shadowing device.

For example, the shadowing device is detachable. A detachable shadowing device allows for one or more of different ear shapes and different ear sizes of different users. A user can select the one or more of a shape and a size.

By keeping the light shadowed or hidden, attention is kept on the skin illuminated flesh instead of on the reflected light, also preventing dazzling a bystander.

Alternatively, or additionally, the shadowing device is substantially translucent. For example, the shadowing device comprises translucent material that is colored one or more of red and orange. Thereby, the flesh-illuminating device optically blends with the illuminated user's ear.

For example, an illuminated section of the shadowing device is reflective so that the light reflected off the user's skin can be reflected back to the user's skin. For example, the color of the shadowing device is one or more of the same as and similar to a color of the user's ear.

For example, the power source is physically attached to the flesh-illuminating device. For example, the controller is physically attached to the flesh-illuminating device. For example, the power source comprises a sourcing cable. For example, the controller comprises a sourcing cable. For example, the sourcing cable transmits one or more of a signal and power. For example, the signal comprises an audio signal. For example, the power source comprises an optic fiber. For example, the controller comprises an optic fiber.

If the flesh-illuminating device also has audio functionality, the sourcing cable can be attached to the earbuds, so that the sourcing cable is visible in front of the user's head. Alternatively, the sourcing cable can be located behind the ear.

For example, the flesh-illuminating device is mounted on the shadowing device. For example, the shadowing device comprises the light source. For example, the shape holder comprises the shadowing device. For example, the flesh-illuminating device does not comprise the shape holder.

For example, the flesh-illuminating device comprises a fiber optic cable. For example, the flesh-illuminating device comprises a fiber optic cable that is illuminated by one or more of blue light, ultraviolet light and laser light. For example, the fiber optic cable is shielded up to a base of the flesh-illuminating device. At the base, phosphor is applied so as to convert the light after excitation of the phosphor to one or more of white light, orange light, red light, and another wavelength of light, and a combination of wavelengths. The phosphor can be one or more of applied, painted, mounted, injected, and molded.

For example, the flesh-illuminating device comprises one or more of phosphor and remote phosphor. For example, the shadowing device comprises one or more of phosphor and remote phosphor. For example, the flesh-illuminating device comprises one layer of phosphor, one reflective layer, and one shadowing layer. For example, the phosphor layer is engineered so that it is in contact with the fiber optic cable.

For example, the sensor comprises one or more of an ambient light sensor, a pulse sensor, a vibration sensor, a touch sensor, a microphone, an accelerometer sensor, and another sensor.

For example, the sensor comprises a touch sensor configured to determine when the flesh-illuminating device does one or more of contact skin of the user and come within a predetermined distance of the user.

For example, the flesh-illuminating device only illuminates locations where the touch sensor determines that the flesh-illuminating device does one or more of contact skin of the user and come within a predetermined distance of the user.

For example, the touch sensor can be used to activate the light source in an area where the flesh-illuminating device touches the user's skin. For example, the touch sensor can be used to lower an intensity of the light source where the flesh-illuminating device touches the user's skin, so that the flesh is more evenly illuminated.

For example, the sensor is part of a free-standing device not comprised in the flesh-illuminating device. For example, the free-standing device comprises one or more of a smart phone, a smart watch, a smart wearable, a smart device, a tablet, and another free-standing device. For example, data generated by the sensor is transmitted to the flesh-illuminating device. For example, the sensor data is transmitted wirelessly. For example, the wireless transmission occurs using one or more of Bluetooth, Zigbee, WiFi, near-field communication (NFC), and another wireless transmission method.

For example, the ambient light sensor is used to regulate an intensity of the light source.

For example, the pulse sensor is used to make the flesh-illuminating device pulse in approximate synchronization with a heartbeat rhythm generated by the pulse sensor.

For example, the vibration sensor is used to power the flesh-illuminating device in synchronization with movement of a user's body. For example, the vibration sensor comprises a larynx sensor configured to detect vibration of the user's larynx. For example, the larynx sensor can detect a user doing one more of growling, talking, screaming, laughing, and making another sound, and can then transmit sensor data to the flesh-illuminating device. The sensor data can then be used to control the light source.

For example, the flesh-illuminating device further comprises a built-in sound-emitting device, and the microphone is used as a hands-free sensor. For example, the microphone can detect a user doing one more of growling, talking, screaming, laughing, saying a desired phrase, and making another sound, and can then do one or more of power up, light up, control the flesh-illuminating device, generate a sound, and take another desired action. For example, the desired phrase comprises one or more of “I love you” and “Will you marry me?”

For example, the sensor comprises an accelerometer sensor usable to control the light source in synchronization with movement of a user's body, for example, one or more of jogging, running, bicycling, skating, skiing, dancing, and another body movement.

For example, one or more of the power source, the controller, the user interface and the sensor forms a horseshoe or neckband shape configured to be worn around the user's neck.

For example, one or more of the power source, the controller, the user interface and the sensor forms a diadem shape, so that the flesh-illuminating device can hold up a person's hair, exposing the user's ear.

For example, one or more of the light source, the shape holder, the shadowing device, and the sound-emitting device comprises the power source. For example, one or more of the light source, the shape holder, the shadowing device, the controller, and the sound-emitting device comprises the user interface. For example, one or more of the light source, the shape holder, the shadowing device, and the sound-emitting device comprises the sensor.

For example, another device can comprises the power source. For example, another device comprises the controller. For example, another device comprises the user interface. For example, another device can comprises the sensor. For example, the other device comprises one or more of a cell phone, a tablet, a media player, and another device.

For example, a small container comprises the power source. For example, a small container comprises the controller. For example, a small container comprises the user interface. For example, a small container comprises the sensor. For example, the small container can be carried by a user, or placed in a user's pocket, or clipped to a user's clothes.

For example, the flesh-illuminating device further comprises a reflective surface. The reflective surface reflects light towards the user's flesh.

For example, the flesh-illuminating device further comprises optics. For example, the optics can change an angle of light distribution. For example, the optics can narrow a cone of light from an approximate 160 degree angle of distribution to an approximate 40 degree angle of distribution.

For example, the flesh-illuminating device illuminates a user's ear. For example, the flesh-illuminating device illuminates a back part of a user's ear where the ear attaches to the head. For example, the ear is illuminated by the flesh-illuminating device, which is placed between the ear and the head, in a channel formed by an intersection of the back part of the ear and the head. These embodiments provide optimal illumination of a majority of a surface of the ear.

For example, the light source goes around the base of the ear.

For example, the sound-emitting device comprises earbuds that are illuminated so as to illuminate a user's ear canal. For example, the sound-emitting device comprises radially mounted lights, the sound-emitting device configured to illuminate the ear around the ear canal. If the sound-emitting device comprises earbuds having a more complicated shape, the light source may be positioned so as to illuminate an area near an edge of the earbud, so that the light source illuminates the flesh of the central part of the ear, but is not directly visible outside the earbuds.

For example, the flesh-illuminating device is placed at an inner side of one or more of a choker, necklace, medallion, and pendant worn by the user, so as to illuminate the flesh. For example, the light source is located around an edge of the choker.

For example, the flesh-illuminating device comprises a headband so that a user placing the headband in their hair lights up one or ore of their hair, ears and skin. For example, the flesh-illuminating device can be positioned in a root of artificial hair so that the hair is glowing.

In the case of a device comprising a sound-emitting device, the sourcing cable may be attached to the earbuds. In such cases, alternatively, or additionally, the sourcing cable may be positioned behind the user's ears.

For example, the flesh-illuminating device illuminates the user's earlobes.

For example, the flesh-illuminating device comprises an earring. For example, the earring is approximately shaped like a button. For example, the earring comprises a pin that goes through the user's ear. For example, the earring clips onto the user's ear. For example, the earring comprises one or more of the power source, the light source, the battery, the controller, and the user interface. For example, the light source could be placed on a side of the button near the earlobe. For example, the light source could be part of the earring pin.

For example, one or more of the power source, the light source, the battery, the controller, and the user interface is embedded in a pendant attached to the earring.

For example, the light source emits light using decorative earring stoppers. For example, the earring stoppers are one or more of exchangeable and replaceable. For example, the light source emits light from the button part of the earring.

For example, the earring can emit light. For example, the earring can surround the user's ear, illuminating the helix of the ear. For example, the button earring also serves decorative functions as well as illuminating the helix of the ear.

For example, the flesh-illuminating device comprises an earplug that is attached to the earlobe by stretching the earlobe. For example, the light source at a radial periphery of the ear so as to best illuminate the earlobe. For example, one or more of the power source, the light source, the battery, the controller, and the user interface is placed in an empty space formed by the flesh-illuminating device.

For example, the flesh-illuminating device comprises a decorative earring. For example, the flesh-illuminating device comprises a dragon earring. For example, the flesh-illuminating device comprises a dragon earring that is positioned at the front part of the ear helix. For example, the light source is placed at the back of the ear around the ear helix. Such an embodiment has a dual purpose of beautiful ornamental earrings and a light source along the ear helix.

For example, the flesh-illuminating device comprises a patch tattoo. For example, the tattoo comprises a substantially transparent, flexible material. For example, the tattoo comprises one or more of silicone and thermoplastic elastomer (TPE).

For example, the light source is placed on a user's belly button ring on the flesh side, so as to illuminate the user's flesh surrounding the belly button.

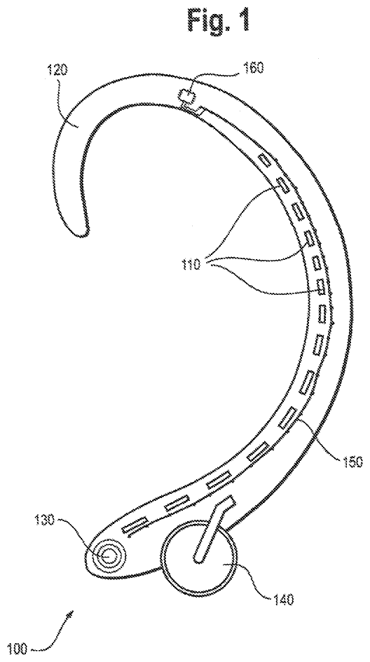

FIG. 1 is a drawing of a flesh-illuminating device 100. The flesh-illuminating device 100 comprises a light source 110 configured to generate light. As depicted, the flesh-illuminating device 100 comprises multiple light sources 110. Alternatively, the flesh-illuminating device may comprise only one light source.

The light source 110 is configured to generate light usable to illuminate flesh of a user (not shown). For example, the light source 110 is configured to generate light usable to illuminate the flesh of a human user. Alternatively, or additionally, the light source 110 is configured to generate light usable to illuminate the flesh of a non-human user.

For example, the light source 110 comprises one or more of a light-emitting diode (LED), an organic light-emitting diode (OLED), a micro-LED, a nano-LED, an LED filament, a laser, a fiber optic light source, a glowing fiber optic light source, a filament light bulb, a non-filament light bulb, a fluorescent light source, a future light source, bioluminescence, chemiluminescence, phosphorescence, radio-luminescence, photoluminescence, electroluminescence, and another light source. As depicted, the light sources 110 each comprise an LED. For example, the light sources 110 are positioned so that a beam angle of the emitted light points in a general direction of the back part of the ear of the user (not shown).

The flesh-illuminating device 100 further comprises a shape holder 120 operably connected to the light source 110, the shape holder 120 configured to hold the light source 110 in place near the user's flesh to be illuminated. In this example, the shape holder 120 is configured to hold the light source 110 in place so that the light source 110 can illuminate the user's flesh. For example, the shape holder 120 is configured to hold the light source 110 in place so that the light source is near the flesh of the user's ear. For example, the shape holder 120 is configured to hold the light source 110 in place at an approximate distance from the flesh of the user's ear of between approximately zero and approximately 20 millimeters (mm).

For example, the shape holder 120 holds the light source 110 in a desired shape. For example, the shape holder 120 is flexible. For example, the shape holder 120 is inflexible. For example, the shape holder 120 holds the light source 110 in a curved shape of the user's ear where the ear connects to the head. For example, the shape holder 120 has one or more of a shape and a curvature generally following a shape of the ear.

The flesh-illuminating device 100 further comprises a user interface 130 operably connected to the light source 110, the user interface 130 configured to allow the user to control the light source 110. For example, the user interface allows the user to adjust one or more of an amount of light emitted by a light source 110, power used by the flesh-illuminating device 100, which light sources 110 are turned on, which light sources 110 are turned off, a degree of illumination provided by a given light source 110, a color of light provided by a given light source 110.

For example, the user interface 130 comprises one or more of a button and a switch. For example, the user interface 130 comprises a touch interface.

The flesh-illuminating device further comprises a power source 140 operably connected to the light source 110. As depicted, the power source 140 comprises a battery. Optionally, the controller 150 comprises the power source 140. Alternatively, the power source 140 comprises the controller 150.

For example, the flesh-illuminating device 100 further comprises a controller 150 that controls operation of the user interface 130 and comprises needed software. For example, the controller 150 comprises a printed circuit board (PCB). The controller 150 connects the light sources 110 to the user interface 130. The controller 150 also electrically connects the light sources 110 to the power source 140. For example, the controller 150 comprises a sourcing cable. For example, the controller 150 comprises a smart device (not shown). For example, the smart device comprises one or more of a laptop, a smart watch, a smart phone, a tablet, a computer, and another smart device.

For example, the flesh-illuminating device 100 further comprises a microprocessor 160. The controller 150 connects the microprocessor 160 to one or more of the light sources 110. The microprocessor 160 is configured to perform one or more of dimming, power regulation, switching inputs, and switching outputs. The controller 150 further connects the user interface 130 to one or more of the light sources 110, enabling user control of the light sources 110.

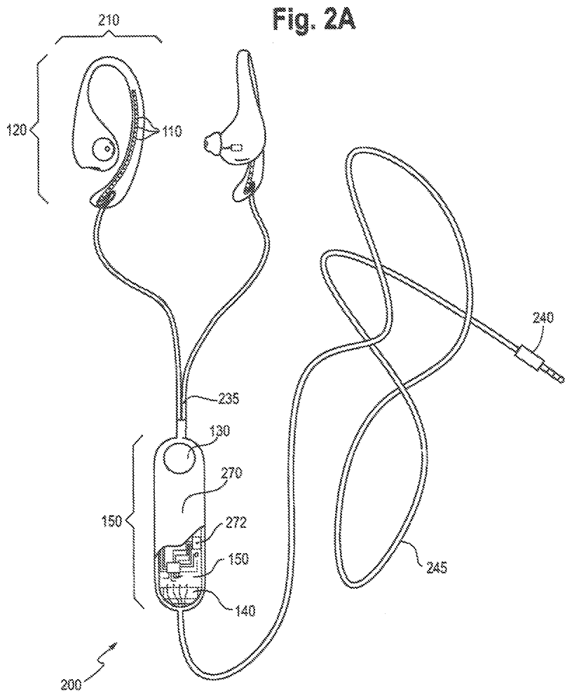

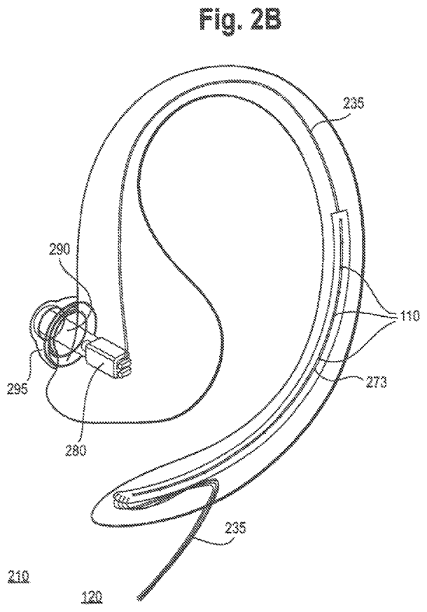

FIGS. 2A-2B are a set of two drawings of a flesh-illuminating device 200 comprising a sound-emitting device 210 configured to emit sound. The sound-emitting device 210 comprises an earphone 210 suitable for emitting sound audible to an ear of the user (not shown). In this example, the shape holder 120 is coterminous with the earphone 210.

As shown in FIG. 2A, the flesh-illuminating device 200 further comprises a light source 110. In this example, the flesh-illuminating device 200 comprises multiple light sources 110. For example, the light sources 110 comprise micro-Light Emitting Diodes (micro-LEDs). For example, the micro-LEDs 110 are positioned so that a beam angle of the emitted light points in a general direction of the back part of the ear of the user (not shown).

The flesh-illuminating device 200 further comprises the shape holder 120 operably connected to the light sources 110, the user interface 130 operably connected to the light sources 110, the power source 140, the controller 150, the shape holder wire 235, an audio interface 240, and an audio interface wire 245. In this example, the controller 150 comprises the power source 140. For example, the power source 140 comprises a battery. For example, the controller 150 sends power to the light sources 110 using pulse width modulation (PWM) using a frequency of at least 15 kilohertz (kHz) in order to reduce electrical noise created by interference between the audio signal generated by the audio interface 240 and the signal generated by the controller 150 to power up the light sources 110. The power source 140 sends power to the light sources 110. For example, the power source 140 sends power to the light sources 110 using pulse width modulation (PWM).

The controller 150 is operably connected to the earphone 210 using the shape holder wire 235. The audio interface 240 comprises an audio connector configured to do one or more of receive and transmit an audio signal. The audio interface 240 comprises one or more of a digital audio interface 240 and an analog audio interface 240. For example, and as shown, the audio interface 240 comprises an analog 3.5 millimeter (mm) stereo jack comprising a microphone pin as a fourth pin. For example, a microphone is also usable as a remote control. Alternatively, or additionally, the audio interface 240 comprises one or more of a “Lightning” audio interface manufactured by Apple, Inc. of Cupertino, Calif. (www. apple. com) and a Universal Serial Bus-C (USB-C) audio interface. For example, the audio interface also can be used as a power source 140.

For example, the user interface 130 comprises one or more of an interface switch and a touch interface. The user interface 130 further comprises the controller 150. The controller 150 directs operation of the user interface 130 and comprises needed software. The user interface 130 is protected by a user interface housing 270.

An audio signal received by the audio interface 240 is transmitted over the audio interface wire 245 and is received by the controller 150. The controller 150 then optionally processes the signal. The controller 150 then passes the audio signal through to the shape holder wire 235 and on to the earphone 210. The controller 150 further comprises an optional universal serial bus (USB) port 272. The USB port 272 is usable for one or more of charging the power source 140, communication, firmware upgrade, software upgrade, and other purposes.

FIG. 2B is a blown up figure showing details regarding the sound-emitting device 210, which as previously mentioned, is in this example coterminous with the shape holder 120. The shape holder 120 comprises the light sources 110. The shape holder 120 holds the light sources 110 in position around a back side of a user's ear (not shown). The shape holder 120 further comprises a flexible printed circuit (FPC) 273 providing conductive connections between the light sources 110, the controller 150 and the power source 140. The FPC 273 comprises the light sources 110. For example, the FPC 273 is suspended inside the shape holder 120. For example, the FPC 273 is positioned so that the light sources 110 emit light toward the back of the user's ear. The shape holder wire 235 runs through the length of the shape holder 120.

The shape holder 120 is generally shaped to match the contours of the ear of the user (not shown). For example, the shape holder 120 comprises material having a substantially solid color. For example, the shape holder 120 comprises material having one or more of a slit or hole. For example, the shape holder 120 comprises material that is one or more of transparent, translucent and semi-transparent, thereby allowing the light emitted by the light sources 110 to emanate from the shape holder 120 in a direction of the ear.

For example, the sound-emitting device 210 further comprises a balanced armature driver 280 operably connected to the light sources 110 via the shape holder wire 235, the balanced armature driver 280 configured to receive an audio signal received from the audio interface 240 (not shown in FIG. 2B). The balanced armature driver 280 is further configured to generate a modified audio signal. The sound-emitting device 210 further comprises an acoustic channel 290 configured to transmit the modified audio signal from the balanced armature driver 280. The sound-emitting device 210 further comprises an optional eartip 295, the eartip 295 configured to snugly fit into the user's ear (not shown).

The balanced armature driver is operably connected via the shape holder wire 235 to the acoustic channel 290.

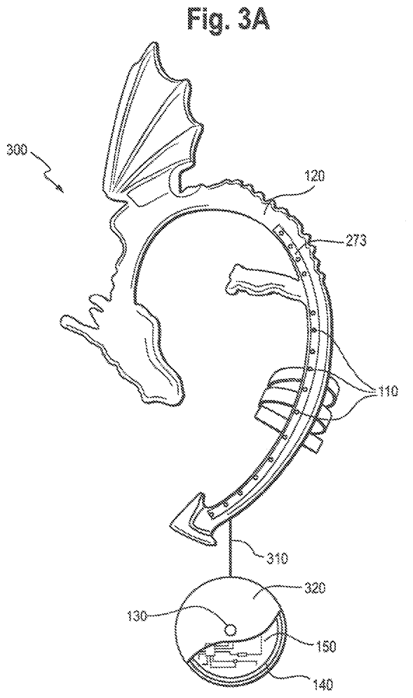

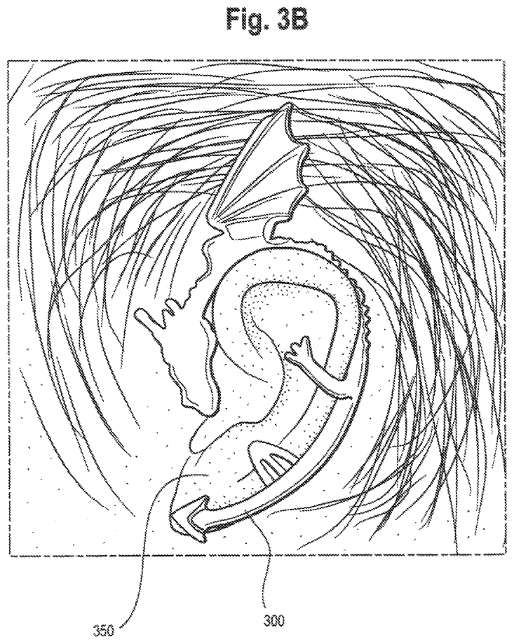

FIGS. 3A-3B are a set of two drawings of a flesh-illuminating device 300 designed to be worn around an ear of a user. For example, the flesh-illuminating device 300 can be worn around a helix of the user's ear.

FIG. 3A depicts components of the flesh-illuminating device 300. In this example, the flesh-illuminating device 300 comprises multiple light sources 110. For example, and as depicted, the light sources 110 comprise LEDs. The flesh-illuminating device 300 further comprises the shape holder 120 operably connected to the light sources 110, the shape holder configured to hold the flesh-illuminating device 300 in place around the ear. The flesh-illuminating device 300 further comprises the user interface 130, the user interface 130 operably connected to the light sources 110 via an interface-shape holder wire 310, the user interface 130 configured to allow the user to control the light sources 110.

The flesh-illuminating device 300 further comprises the power source 140, the power source 140 operably connected to the shape holder 120 via the user interface-shape holder wire 310. In this example, the power source 140 comprises the controller 150. The flesh-illuminating device 300 further comprises the controller 150. The controller 150 directs operation of the user interface 130 and comprises needed software. A power source housing 320 comprises the power source 140, the user interface 130, and the controller 150. For example, the power source 140 comprises a battery. The power source 140 sends power to the light sources 110. For example, the power source 140 sends power to the light sources 110 using pulse width modulation (PWM). For example, the power source 140 sends power to the light sources 110 using direct current (DC).

The flesh-illuminating device 300 further comprises the FPC 273 providing conductive connections between the light sources 110, the controller 150 and the power source 140. The FPC 273 comprises the light sources 110. For example, the FPC 273 is suspended inside the shape holder 120. For example, the FPC 273 is positioned so that the light sources 110 emit light toward the back of the user's ear.

FIG. 3B depicts the ear-illuminating device 300 being worn on the ear 350 of a human user.

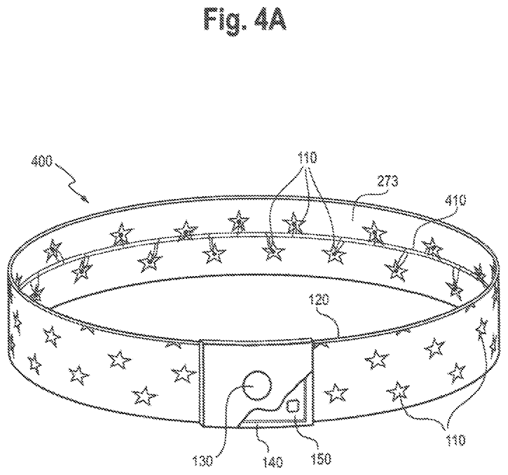

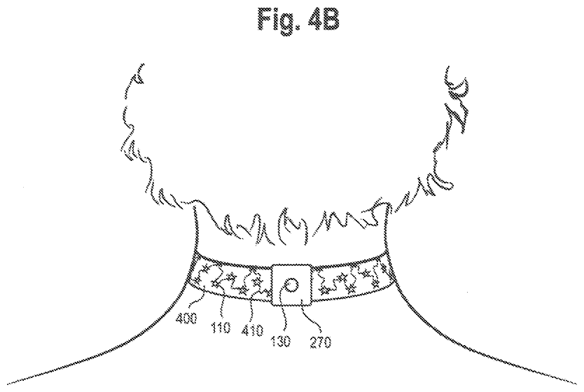

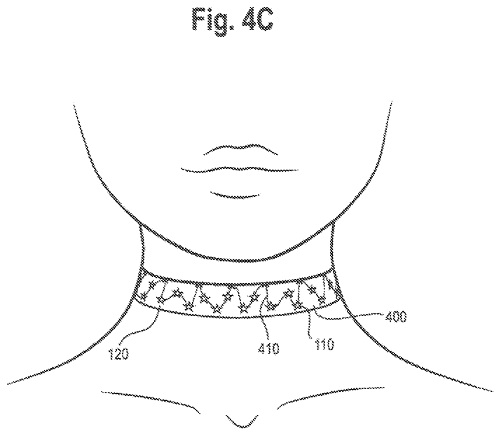

FIGS. 4A-4C are a set of three drawings of a flesh-illuminating device designed to be worn as a band around a neck of a user.

FIG. 4A depicts a drawing of a flesh-illuminating device 400 designed to be worn as a band around a user's neck at a time when it is not being worn on the user's neck. The flesh-illuminating device 400 comprises a plurality of light sources 110. In this example, the light sources 110 comprise micro-LEDs. In this example, the light sources 110 comprise lighted ornaments. In this example, the light sources 110 comprise star-shaped lighted ornaments 110. The star-shaped lighted ornaments 110 comprise one or more of semi-transparent material and opaque material. The star-shaped lighted ornaments 110 also function as a shadowing device configured to reduce one or more of direct reflection of light from the user's flesh and direct light from the light sources 110.

In this example, the shape holder 120 is coterminous with the flesh-illuminating device 400. Alternatively, or additionally, embodiments of the invention can be worn on one or more of an ankle of a user and a wrist of a user. For example, the flesh-illuminating device 400 comprises silicone. For example, the silicone is flexible. For example, the silicone is substantially transparent. For example, the flesh-illuminating device comprises thermoplastic elastomer (TPE).

In this example, the flesh-illuminating device 400 is coterminous with the shape holder 120. Also shown are the plurality of light sources 110, the controller 150, the user interface 130, the power source 140, and conductive tracks 410 by which the power source 140 is operably connected to the light sources 110. The conductive tracks 410 may be suspended inside the flesh-illuminating device 400.

The power source 140 further comprises the controller 150 that directs operation of the user interface 130 and comprises needed software. The flesh-illuminating device 400 further comprises the flexible printed circuit (FPC) 273 providing conductive connections between the light sources 110, the controller 150 and the power source 140. In this example, the power source 140 comprises the controller 150.

FIG. 4B depicts a drawing of a back view of the flesh-illuminating device 400. Also shown again are the plurality of light sources 110. The flesh-illuminating device 400 is coterminous with the shape holder 120. Also shown again is the user interface 130. Also shown is the user interface housing 270. Also shown are the conductive tracks 410 configured to conduct electricity from the power source 140 to the light sources 110. Also shown is the user interface 130, which is operably connected to the light sources 110 by the conductive tracks 410. The star-shaped lighted ornaments 110 also function as a shadowing device configured to reduce one or more of direct reflection of light from the user's flesh and direct light from the light sources 110. The power source 140 can be controlled wirelessly. The power source 140 can be controlled using a button comprised in the user interface 130.

FIG. 4C depicts a drawing of a front view of a neck of a user wearing a flesh-illuminating device 400 that is again coterminous with the shape holder 120. Also shown again are the light sources 110 and the conductive tracks 410.

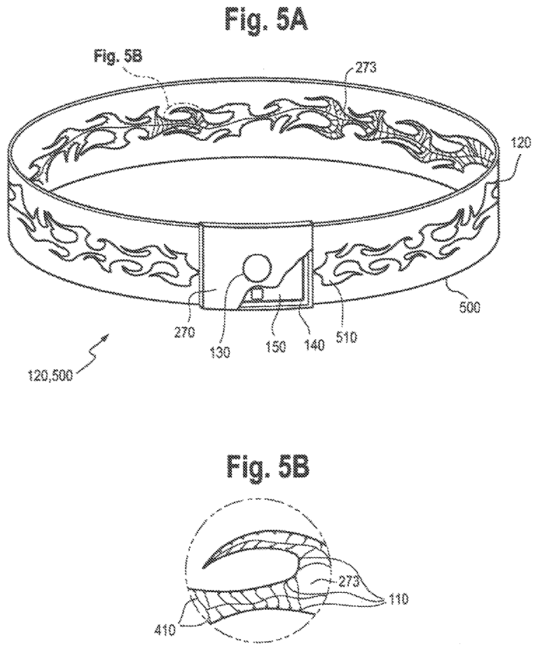

FIGS. 5A-5B are a set of two drawings of a flesh-illuminating device 500 designed to be worn as a band around a user's neck, FIG. 5A shows the flesh-illuminating device 500 at a time when it is not being worn on the user's neck. In this example, the shape holder 120 is coterminous with the flesh-illuminating device 500. Alternatively, or additionally, embodiments of the invention can be worn on one or more of an ankle of a user and a wrist of a user. For example, the flesh-illuminating device 500 comprises silicone. For example, the silicone is flexible. For example, the silicone is substantially transparent. For example, the flesh-illuminating device comprises thermoplastic elastomer (TPE).

The flesh-illuminating device 500 comprises a plurality of light sources 110 (shown in FIG. 5B). In this example, the light sources 110 comprise micro-LEDs. The flesh-illuminating device further comprises the user interface 130, the power source 140, and the controller 150. For example, the user interface 130 comprises one or more of a button and a switch. In this example, the flesh-illuminating device 500 further comprises a light shadowing device 510. In this example, the light shadowing device 510 comprises a light shadowing ornament 510. In this example, the light shadowing device 510 comprises a flame-shaped light shadowing ornament 510.

The flesh-illuminating device 500 further comprises the FPC 273 providing conductive connections between the light sources 110, the power source 140, and the controller 150.

FIG. 5B is a blown-up drawing of the light shadowing device 510 comprising the FPC 273. The light shadowing device 510 comprises the light sources 110. Preferably, although not necessarily, the light sources 110 comprise micro-LEDs. Preferably, but not necessarily, the FPC 273 has a predominantly clear color. Alternatively, or additionally, the FPC 273 has a shape that generally follows a shape of the light shadowing device 510.

The FPC 273 comprises conductive tracks 410 by which the power source 140 is operably connected to the light sources 110. The FPC 273 may be suspended inside the flesh-illuminating device 500. The light shadowing device 510 comprises one or more of semi-transparent material and opaque material. The light shadowing device 510 is configured to reduce one or more of direct reflection of light from the user's flesh and direct light from the light source 110.

The power source 140 further comprises the controller 150 configured to facilitate operation of the flesh-illuminating device 500.

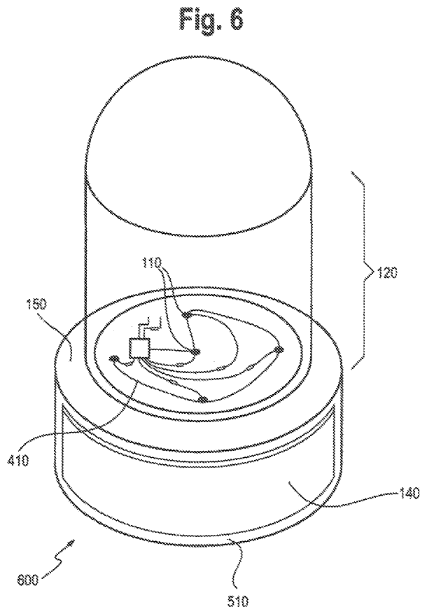

FIG. 6 is a drawing of a flesh-illuminating device 600 designed to be worn inside the nostril of a user. The flesh-illuminating device 600 comprises the light sources 110, the shape holder 120, the battery 140, the controller 150, the conductive tracks, and the conductive tracks 410. Preferably, although not necessarily, the light sources 110 comprise micro-LEDs. Preferably, although not necessarily, the shape holder 120 comprises a material that is one or more of predominantly clear and predominantly translucent material.

Optionally, and as depicted, the power source 140 is coterminous with the light shadowing device 510. The flesh-illuminating device 500 comprises a plurality of light sources 110. In this example, the light sources 110 comprise micro-LEDs.

The light shadowing device 510 further comprises the conductive tracks 410 by which the power source 140 is operably connected to the light sources 110.

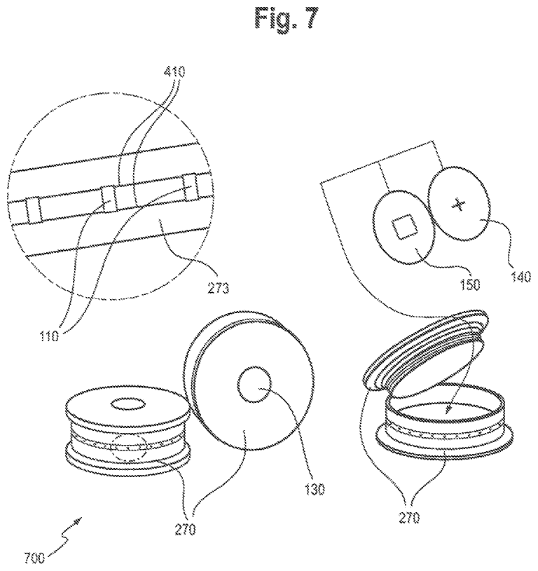

FIG. 7 is a drawing of a flesh-illuminating device 700 designed to be worn as an ear gauge on an earlobe of a user. The flesh-illuminating device 700 comprises the light sources 110, the shape holder 120 which in this example is coterminous with the user interface housing 270, the user interface 130, the power source 140, the controller 150, the FPC 273, and the conductive tracks 410. The drawing shows three examples of the shape holder 120. Preferably, although not necessarily, the light sources 110 comprise micro-LEDs. For example, the user interface 130 comprises one or more of a button and a switch.

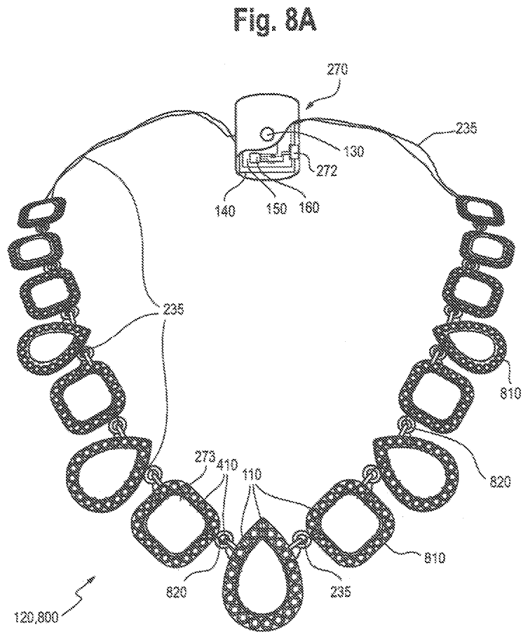

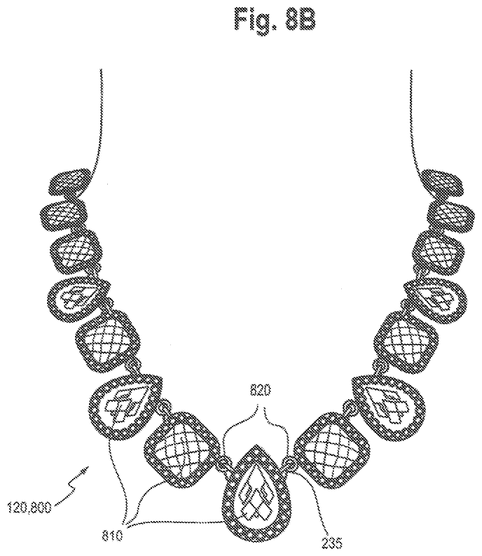

FIGS. 8A-8B are a set of two drawings of a flesh-illuminating device 800 designed to be worn as a necklace on a neck of a user. FIG. 8A depicts a drawing of a back view of the flesh-illuminating device 800. The flesh-illuminating device comprises a number of necklace ornaments 810. For example, the necklace ornament 810 comprises a gem 810. The necklace ornament 810 are connected by necklace wires 820.

In this example, the flesh-illuminating device 800 is coterminous with the shape holder 120. The flesh-illuminating device 800 comprises the light sources 110, the user interface 130, the power source 140, the controller 150, the microprocessor 160, the shape holder wire 235, the controller housing 270, the USB port 272, the FPC 273, and the conductive tracks 410. The shape holder wire 235 electrically connects a necklace ornament 810 to one or more of an adjacent necklace ornament 810 and the controller housing 270. Preferably, although not necessarily, the light sources 110 comprise micro-LEDs. For example, the controller 150 comprises one or more of a button and a switch (depicted as user interface 130 here).

FIG. 8B depicts a drawing of a front view of the flesh-illuminating device 800 as worn on the neck of a human user. The flesh-illuminating device 800 is coterminous with the shape holder 120. Fewer elements of the flesh-illuminating device are visible in the front view shown in FIG. 8B. Visible are the shape holder wire 235, the necklace ornaments 810, and the necklace wires 820.

For example, it will be understood by those skilled in the art that software used by the flesh-illuminating device and method for lighting flesh may be located in any location in which it may be accessed by the flesh-illuminating device. It will be further understood by those of skill in the art that the number of variations of the method and device are virtually limitless. It is intended, therefore, that the subject matter in the above description shall be interpreted as illustrative and shall not be interpreted in a limiting sense.

While the above representative embodiments have been described with certain components in exemplary configurations, it will be understood by one of ordinary skill in the art that other representative embodiments can be implemented using different configurations and/or different components. For example, it will be understood by one of ordinary skill in the art that the order of certain steps and certain components can be altered without substantially impairing the functioning of the invention.

The representative embodiments and disclosed subject matter, which have been described in detail herein, have been presented by way of example and illustration and not by way of limitation. It will be understood by those skilled in the art that various changes may be made in the form and details of the described embodiments resulting in equivalent embodiments that remain within the scope of the invention. It is intended, therefore, that the subject matter in the above description shall be interpreted as illustrative and shall not be interpreted in a limiting sense.

We claim:

1. A flesh-illuminating device, comprising:
a light source configured to generate light usable to illuminate flesh of a user;
a shape holder operably connected to the light source, the shape holder configured to hold the light source in place so that the light source can illuminate the user's flesh;
a user interface operably connected to the light source, the user interface configured to allow the user to control the light source;
and a power source operably connected to the light source, the power source configured to provide power to the flesh-illuminating device.

2. The flesh-illuminating device of claim 1, wherein the light source is configured to generate light usable to illuminate flesh of a human user.

3. The flesh-illuminating device of claim 1, wherein the flesh-illuminating device provides nearly 360 degrees of visibility of the illuminated flesh.

4. The flesh-illuminating device of claim 1, further comprising a shadowing device configured to reduce one or more of direct reflection of light from the user's flesh.

5. The flesh-illuminating device of claim 4, wherein the shadowing device is physically connected to one or more of the light source and the shape holder.

6. The flesh-illuminating device of claim 1, wherein the flesh-illuminating device is configured to send the user a reminder to turn the light source on for one or more of safety reasons, pre-set user preferences, and other reasons.

7. The flesh-illuminating device of claim 6, wherein the flesh-illuminating device is configured to receive a user preference regarding a user activity that enables the flesh-illuminating device.

8. The flesh-illuminating device of claim 7, wherein the user activity comprises one or more of running, bicycling, hiking, skating, skiing, participating in a designated sport, and walking.

9. The flesh-illuminating device of claim 1, the flesh-illuminating device configured to activate based upon occurrence of one or more activation criterion.

10. The flesh-illuminating device of claim 9, wherein the activation criterion comprises one or more of a location of the flesh-illuminating device, a distance of the flesh-illuminating device from the user, a detection of ambient light, a date, a location, and a time of day.

11. The flesh-illuminating device of claim 9, wherein the controller receives the activation criterion from the user.

12. The flesh-illuminating device of claim 9, wherein a smart device to which the controller is connected receives the activation criterion from the user.

13. The flesh-illuminating device of claim 9, wherein the flesh-illuminating device is configured to enter an activated mode within a predetermined distance from a location of interest.

14. The flesh-illuminating device of claim 13, wherein the location of interest comprises one or more of a crosswalk, an intersection, a highway, and a major road.

15. The flesh-illuminating device of claim 13, wherein the activated mode comprises one or more of a stronger light, a weaker light, a blinking light and another attention-drawing mode of the light source.

16. The method of claim 15, wherein the activated mode is configured to attract attention of a driver of a motor vehicle.

17. The flesh-illuminating device of claim 1, wherein the flesh comprises one or more of a human ear, a human ear root, a human ear lobe, an earring worn by a human, a human ear edge, a human nose, a human mouth, a human hand, a human finger, a human fingernail, a human toenail, a human foot, a human toe, a human head, a human neck, a human breast, human buttocks, a human buttocks cheek, a human cheek, a human stomach, a human belly button, a human ankle, a bra worn by a human, a belt worn by a human, a wristband worn by a human, a wristwatch worn by a human, a sandal worn by human, a shoe worn by human, another clothing item worn by a human, a human body, human flesh, a human lip, and another human body part.

18. The flesh-illuminating device of claim 1, further comprising a sensor configured to determine when the flesh-illuminating device does one or more of contact skin of the user and come within a predetermined distance of the skin of the user.

19. The flesh-illuminating device of claim 1, wherein the flesh-illuminating device further comprises a sound-emitting device configured to emit sound.

20. A flesh-illuminating device, comprising:
a light source configured to generate light usable to illuminate flesh of a human user;
a shape holder operably connected to the light source, the shape holder configured to hold the light source in place so that the light source can illuminate the user's flesh;
a user interface operably connected to the light source, the user interface configured to allow the user to control the light source;
a power source operably connected to the light source, the power source configured to provide power to the flesh-illuminating device;
and a sensor configured to determine when the flesh-illuminating device does one or more of contact skin of the user and come within a predetermined distance of the user, wherein the flesh-illuminating device is configured to enter an activated mode within a predetermined distance from a location of interest, wherein the location of interest comprises one or more of a crosswalk, an intersection, a highway, and a major road,
wherein the activated mode comprises one or more of a stronger light, a weaker light, a blinking light and another attention-drawing mode of the light source,
wherein the activated mode is configured to attract attention of a driver of a motor vehicle,
wherein the light source is configured to activate based on an activation criterion received from a user, the activation criterion comprising one or more of a location of the flesh-illuminating device, a distance of the flesh-illuminating device from the user, a detection of ambient light, a date, a location, and a time of day,
wherein the flesh-illuminating device is configured to send a user a reminder to turn the light source on for one or more of safety reasons, pre-set user preferences, and other reasons,
wherein the flesh-illuminating device is further configured to receive a user preference regarding a user activity that enables the flesh-illuminating device, the user activity comprising one or more of running, bicycling, hiking, skating, skiing, participation in a designated sport, and walking.
